(19) United States
(12) Patent Application Publication
(10) Pub. No.: US 2012/0150268 A1
(43) Pub. Date: Jun. 14, 2012

(54) Treatment System for Articulated Joints

(76) Inventors: Brian Doherty, Melbourne, FL (US); John McMorris, Indialantic, FL (US)

(21) Appl. No.: 13/159,112

(22) Filed: Jun. 13, 2011

Related U.S. Application Data
(60) Provisional application No. 61/353,994, filed on Jun. 11, 2010.

(51) Int. Cl. A61F 7/08; A61H 7/00; A61F 7/10

(52) U.S. Cl. 607/108; 601/149

(57) ABSTRACT

A treatment for articulated joint injuries and maladies, whether pre- or post-operative or degenerative in nature, and more specifically to systems and/or devices that facilitate joint recovery by applying constant microclimate cooling/heating and optional pressure to an affected joint in a secure, comfortable and reliable manner, even during repeated joint flexure or movement.

### CROSS REFERENCE TO RELATED APPLICATIONS

This application claims the benefit of provisional patent application Ser. No. 61/353,994, filed with the USPTO on Jun. 11, 2010, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to the treatment of articulated joint injuries and maladies, whether pre- or post-operative or degenerative in nature, and more specifically to systems/devices that facilitate joint recovery by applying constant microclimate cooling or heating and optionally compression to an affected joint in a secure, comfortable and reliable manner, even during repeated joint flexure or movement.

2. Background

As is already known in the art, the knee is the most injury-prone joint of the body and, at the same time, is frequently affected by arthroses since it can easily become mechanically unstable and bears the full weight of the body. Muscular imbalance often causes pains in the anterior kneecap. In persons who practice sedentary professions, the ischioscural musculature is for the most part contracted, drawing the articular capsule rearward, increasing contact pressure in an undesirable manner. Sporting injuries of the knee joint frequently result when the lower leg is twisted while placing weight on the joint—often resulting in lesions of the knee and associated local pain. The complexity of the joint renders exact diagnosis and treatment difficult, often contributing to later degenerative processes.

As is also known, other articulating joints evidence similar problems. Some of the more common articulating joints and maladies are discussed in the next few paragraphs.

Ankles are quite complex, being comprised of both the subtalar joint and the ‘true’ ankle joint (itself comprised of the tibia, which forms the medial portion of the ankle; the fibula, which forms the lateral portion of the ankle; and the talus, underneath)—all bearing full body weight loads and being prone to stress and potential injury under torsional loads.

While shoulder joints do not bear the constant heavy loads borne by either knees or ankles, they sustain motion in many axes and are required to bear high dynamic loads. The two main bones of the shoulder are the humerus and the scapula (shoulder blade). The shoulder joint cavity is cushioned by articular cartilage covering the head of the humerus and face of the glenoid. The scapula extends up and around the shoulder joint at the rear to form a roof called the acromion, and around the shoulder joint at the front to form the coracoid process. The end of the scapula (the glenoid) meets the head of the humerus to form a glenohumeral cavity that acts as a flexible ball-and-socket joint. The joint is stabilized by a ring of fibrous cartilage (the labrum) that surrounds the glenoid. Ligaments and tendons join the bones to surrounding muscles, stabilizing the joint. For instance, four short muscles originate on the scapula and pass around the shoulder where their tendons fuse together to form the rotator cuff. These components of the shoulder, along with the muscles of the upper body, work together to manage the stress experienced by the shoulder through flexure, extension, lifting and throwing. Nature's design of this joint underscores an essential point relating to products seeking to bring thermal and/or pressure treatment to articulated joints: the design of a successful treatment product must contemplate both necessary joint range of motion (both extent and angle) as well as the musculature in the affected region.

Even the elbow joint is more complex than most realize and imposes special considerations for treatment. This joint is essentially a ‘hinge’ joint; comprised of the humerus, ulna and radius. The positioning and interaction of these bones allows for a small amount of rotation as well as hinging. Joint stability is principally provided by the ulnar collateral ligament on the medial side of the elbow. The lateral side of the joint suffers the most common afflictions, sometimes going by the name ‘tennis elbow.’

The wrist is formed where the two bones of the lower arm—the radius and the ulna—meet at the hand. It provides passage to the two major nerves of the hand (the median and ulnar nerves) which run the length of the arm to transmit electrical impulses to and from the brain to create movement and sensation.

One of the more common afflictions of the wrist is Carpal Tunnel Syndrome, a pinching of the median nerve within the wrist—frequently a result of highly repetitive and recurring motion. The carpal tunnel is a bony canal within the palm side aspect of the wrist that allows for the passage of the median nerve to the hand. Pinching or compression of this nerve by the transverse carpal ligament sets into motion a progressively crippling disorder which eventually results in wrist pain, numbness and tingling in the hand, pain consisting of a “pins and needles” feeling at night, and weakness in the grip. The wrist is also subject to cartilage damage (much like the knee), which is often repaired with arthroscopic surgery.

All of these joints can suffer damage from high dynamic loading, overloading in/across the wrong motions/axes, and through repeated repetitive motions commonly develop tendon, muscle and joint related—and even degenerative—problems that can be mitigated by using a suitable articulated joint treatment system. When traumatized, these joints frequently swell and may create further trauma by involving local muscles or tendons.

While the body has additional articulating joints (e.g. the neck), each of which may require different overall system design changes to optimize therapeutic outcome, to one skilled in the art it is apparent that a well designed treatment system ‘approach’ will include variations or parameters that enable the treatment of other articulated body joints that are not discussed herein.

Arthroscopic surgery, often used to repair joint/cartilage problems, has become quite common; indeed, arthroscopic knee surgery has become the most commonly performed orthopaedic procedure in the United States. While this surgery can be quite effective in repairing many kinds of joint, cartilage or tendon damage, it typically has limited utility on degenerative joint conditions.

In addition to degenerative conditions catalyzed by injury, osteoarthritis has become one of the most common joint diseases known to man and afflicts millions of people to a clinically significant degree in the United States alone. While degenerative pathologies vary by person, the principal symptoms suffered by osteoarthritis patients are pain in and around the affected joint and a lessening of joint mobility. Largely because arthroscopic procedures generally deliver limited long-term value in treating degenerative joint conditions (and, in part, because some patients do not favor surgery), osteoarthritis centers have sprung into being to help patients improve affected joint mobility and manage pain. The incidence of this disease generally increases with the age of the patient.

As is already known, diverse treatment protocols and devices have been developed to treat different articulated joint (and associated tendon/musculature) problems. Such treatment protocols may include either cooling or heating the affected area in conjunction with different means of either immobilizing the joint or specifically enabling and managing flexure through range of motion and strength building exercises, whether by brace, prosthetic device or flexible wraps. Some of the associated devices apply gentle pressure—others do not—using carefully shaped bandages or inflated structures. Yet other treatment includes the use of anti-inflammatory agents (such as aspirin) or the intra-articular injection of various materials including corticosteroids or cartilage powder(s).

Cooling treatment is commonly used for acute injuries, especially where swelling is or may become present—providing both short-term pain relief and a reduction of swelling by reducing blood flow to the injured area.

For instance, the R.I.C.E. method is commonly used—RICE stands for “Rest, Ice, Compression and Elevation”—where ice or ‘frozen gel’ packs are used to relieve pain, limit swelling and protect the injured tissues, promoting faster healing. Unfortunately, the RICE method has historically relied upon the use of either ice or ‘frozen gel’ to achieve localized cooling, both of which can cause profound skin and/or tissue damage if left in contact with the body for more than about 20 minutes (proper use of this method cycles cooling packs on and off the affected area). This can be problematic in both medical settings (where staff responsibilities may preclude cycling packs on/off at the necessary intervals) and especially at home—where both inadequate training regarding the risks of thermal over-exposure exacerbated by the body's natural tendency towards adaptation (wherein the body becomes less sensitive to temperature sensations) cause the patient to believe the pack doesn't yet need to be removed/cycled. The incidence of RICE method accidents is high enough that numerous class action lawsuits are being developed around failures of this technique.

Various approaches/equipment have been devised to provide such localized cooling—ranging from a frozen bag of peas held in place by hand to using various cooling packs such as those described in U.S. Pat. Nos. 4,628,932 and 6,470,705 and others. Some embodiments include an outer layer that helps buffer the cold so the user can more comfortably hold the pack against the affected area; others integrate layers to absorb attendant bodily fluids and/or condensate from the cooling bags (which are quite cold). Yet other embodiments include various attachment means, including straps in different configurations so the cooling pack can be held against the knee without being manually held in place or means to lash or wrap the cooling bag or container to the joint.

A common problem amongst such devices is their inability to readily conform to the affected joint (in the case of the patents noted above, the knee joint)—especially during motion. It becomes nearly impossible to use such cooling to reduce swelling while simultaneously operating the joint through range of motion or repetitive exercises.

When the attachment means is lashing or wrapping the cooling pack against the injured joint, joint movement commonly causes the lashing to unravel and/or become looser, potentially aggravating the injury and causing additional pain. While wrapping the joint more tightly can mitigate the risks of loosening, joint pain and potential swelling often rises with increased wrapping tension. As well, properly adjusting the cooling pack to the affected joint can be difficult; adjustments may require the joint to be completely unwrapped and then re-wrapped. These problems are exacerbated by the need to cycle cooling packs off the joint every 20 minutes or so in order to prevent tissue damage associated with over cooling.

U.S. Pat. No. 4,585,003 seeks to remedy these problems by using die-cut flexible materials to more conveniently attach cooling bags—but this approach often creates other problems associated with frequent use because the material may not breath, can irritate the skin (sometimes causing rashes) and accommodates bacterial infestations which can transfer to (and affect) the user's skin. These hygiene problems make it unwise to use a given wrap with more than a single patient. Moreover, most such devices do not allow external access to the ice/gel packs for ready removal or replacement.

U.S. Pat. Nos. 5,728,057 and 5,728,058 develop a flexible web which is designed to accommodate bending of the user's knee joint, and which integrate discrete thermal elements into the web to apply either modest heating or cooling to an injured joint. These wraps are meant to be disposable—so hygiene problems are generally eliminated—and the design helps to address the challenges of wrap ‘bunching’ or other undesired movement during repeated knee flexure. Because of how the thermal elements are incorporated into the design, however, only limited heating or cooling can be applied to the joint, sharply limiting therapeutic value. Long-term thermal treatment can become quite expensive since these webs are designed to support only one-time use. Unintended device movement—and movement of thermal elements away from the intended region(s)—is also a problem with other articulated joints.

Similar problems pertain to devices designed to deliver heat, typically by means of disposable heating packs. Disposable packs based upon iron oxidation, such as described in U.S. Pat. Nos. 4,366,804 and 4,649,895 and 5,046,479 are known. However, most such devices are often bulky, cannot maintain a consistent and controllable temperature, have difficulty staying in place during use and/or cannot be easily incorporated into wraps that conform to the body's contours. Such devices are inconvenient to use on a regular or extended basis because the thermal energy may not be immediately available when needed or released in a controllable manner. Some such devices include risks of topical burns—analogous to the skin/tissue damage associated with ice/gel packs, though caused by heat rather than cold—especially due to the non-linear heat release characteristics of iron oxide heating packs. U.S. Pat. No. 6,048,326 describes a wrap that mitigates some of these conformity and positioning problems, but without addressing the non-linear heat release tendencies.

Various devices have been proposed to address longer-term heating or cooling non-linearity or to create a more consistent localized temperature—some in conjunction with compression. U.S. Pat. No. 5,314,455 sets forth a cuff with a watertight cavity shaped to envelope only the anterior and sides of the knee. This device has therapeutic limits—it does not cover all sections of the knee for which cooling is typically specified—and uses a gravity fed approach to fill the cuff The patient must hold a container above the cuff until it is sufficiently filled and later—once the fluid temperature has changed and the fluid needs to be refreshed—must siphon the contained fluid into a container so the process can be repeated. This process is, at best, frustrating, and may require external assistance. As well, such cuffs limit joint flexure and effectively immobilize the patient.

US Patent Application US 2005/0033390 A1 describes a thermal compression system incorporating a (thermal) fluid input and output line with an external pump that circulates the heating/cooling fluid. This system helps mitigate the challenges of replacing thermal packs on a frequent and repetitive basis and solves some of the problems unaddressed in U.S. Pat. No. 5,314,455, but still requires significant patient or external professional interaction and may significantly limit patient mobility and/or joint flexure. It is unclear whether sensors or safety systems exist to limit thermal fluid temperatures to a regime safe for long-term skin/tissue exposure.

Various commercial devices have been developed, as the need for decent treatment devices is so keen. The “Moji” knee, for instance, offers many of the features described in the patents above: frozen gel, a moisture wicking fabric layer, and an elastic wrap which applies tension to help keep the system in place. This design still suffers critical flaws: it must still be removed after 15-20 minutes, (as it is too cold for long-term skin contact), has limited capacity for pressure adjustment, and somewhat limits knee joint flexure.

What is needed is an articulated joint treatment solution that provides:
a constant, consistent and safe microclimate temperature—one chosen by a treatment professional to optimize therapeutic/recovery outcome—in order to achieve therapeutic benefits without any associated risks of adverse events such as skin/tissue damage through over exposure or long-term use;
usage without requiring proximal connection (such as power cords or tubing used to convey either gas or liquid) to ancillary systems, which may substantially limit patient mobility;
a shape/fit that delivers thermal treatment where it is physically needed to enhance joint recovery and comfort;
for joint flexure using one or more ‘active hinges’ to interconnect and register the multiple wraps/pieces in a way that enables full range of motion, repetitive motion and strength building exercises without causing the treatment system to move, bunch, create additional patient discomfort or to relocate the thermal treatment away from the specific affected regions needing this treatment;
a skin contact surface that either inhibits bacterial and/or fungal growth which lends itself to cleaning/sterilization;
a skin contact surface or surfaces that minimize patient skin discomfort and/or allergic reactions;
an adjustable fit, so it can be used on patients of different size and/or so device tension can be optimized to patient comfort and/or the chosen recovery regimen;
sufficient durability to be used in more than one treatment session, whether by the patient or a treatment center (that may use the device amongst several patients);
a design comprised only of materials that are safe for use with skin and tissue so that a broken or compromised device doesn't pose additional danger(s) to the wearer. In the case of a system containing fluid(s), for instance (as discussed below), the fluid(s) should be non-toxic, non-caustic and dielectric (to eliminate shock risks if used in conjunction with powered sub-systems);
an optional capacity to temporarily immobilize the articulated joint treatment system as needed (to position the joint precisely as chosen by a treatment professional while still delivering the benefits of constant-temperature treatment) so the beneficial effects of long-term constant temperature microclimate modification can be delivered while the joint is immobilized. The angle of such immobilization may be either adjustable or non-adjustable to suit therapeutic needs; and
an optional capacity to apply pressure, via integrated means (such as one or more pressurized bladders), to apply pressure to a specific treatment area or areas. To be of greatest utility, said pressure(s) should be adjustable and capable of being sustained for long periods of time. Such pressurized bladders may be a permanent feature of the invention's assembly, or may be installed into pockets or other attachment means.

Unfortunately, as noted herein, previous efforts to create articulated joint treatment devices that modify wearer microclimate suffer from profound disadvantages including: significant risk of skin/tissue damage from prolonged exposure to heat or cold, hygienic risks that may exacerbate joint recovery in post-operative scenarios, overly restrictive joint range of motion, severely restricted patient mobility, the tendency to bunch up or “move” thermal treatment cells away from the intended location(s) during joint flexure and exercise, sub-optimal microclimate temperature and thermal consistency (including the need to cycle thermal packs on/off the joint or failure to sustain the localized temperature within an optimal temperature range), or requiring proximal connection to ancillary systems—whether to enable recirculation, power (such as for thermo-electric modules) or other purposes.

While ice, gels and iron oxides—the most common cooling and heating technologies respectively—have been around for generations, constant cooling devices (whether set for single or multiple stable transitions) have not. A constant cooling device could work to keep the operational temperature at (or approximately at) a preset or given temperature. In cooling, for instance, such a device could be elevated well above said ice and or gel associated temperatures, providing all the benefits of cold therapy without the associated risks, such as, frostbite, histamine and aqueous production.

As is known in the art, thermo-electric modules—with suitable feedback sensors or controls (whether external or integral to the module)—are capable of presenting a stable constant temperature microclimate but suffer additional problems such as: high cost, physical inflexibility, difficulty to incorporate into shapes/wraps etc needed to conform to a joint, and the need to provide electricity to power the modules (requiring attachment to ancillary systems and/or creating additional reliability and/or safety risks).

As is also known in the art, there are a wide range of Phase Change Materials (PCMs), that is, substances with a high heat of fusion, which, melting and solidifying at a certain temperature, are capable of storing and releasing large amounts of energy. Thermal energy is absorbed or released when the material changes from solid to liquid, making PCMs a latent heat storage material.

As is also known, while PCM latent heat storage can be achieved through all forms of chemical transition: (solid-solid, solid-liquid, solid-gas and liquid-gas phase change) the only phase change of practical use in most applications is the solid-liquid change. Liquid-gas phase changes are not practical for use as thermal storage due to the large volumes or high pressures required to store the materials when in their gas phase. Liquid-gas transitions do have a higher heat of transformation than solid-liquid transitions. Solid-solid phase changes are typically very slow and have a rather low heat of transformation.

Initially, solid-liquid PCMs behave like sensible heat storage (SHS) materials; their temperature rises as they absorb heat. Unlike conventional SHS, however, when PCMs reach the temperature at which they change phase (their melting temperature) they absorb large amounts of thermal energy at an almost constant temperature. The PCM absorbs heat without a significant rise in temperature until all the material is transformed to the liquid phase. When the ambient temperature around a liquid material falls, the PCM solidifies, releasing its stored latent heat. These properties make PCMs suited to providing either articulated joint therapeutic system heating or cooling, provided the PCM temperature(s) is/are properly chosen.

The best-known phase change material is water—which can exist as either liquid or ice at 32° F. (0° C.) at normal pressures. Certain properties of water/ice, however, render it of little use (or useless) in given applications, including: the phase temperature cannot be modified (ice is too cold to be used for long in most biological applications as applying ice to tissue quickly results in severe vasoconstriction and vastly reduced capillary blood flow), the water to ice transition results in a volumetric expansion of ˜9%—making it a challenge to use in mechanical applications, and ice exhibits little mechanical “give” in the fully frozen state. While this expansion factor may not be relevant in some joint treatment applications, the overly cold temperature is.

Other PCMs can be either organic or inorganic, can be chemically stable or unstable, can be caustic or non-caustic, flammable/inflammable, etc. In short, like any other substances, PCM chemical properties vary as a function of the specific substance. PCMs are typically characterized by their Heat of Fusion (measured in kJ/kg), the amount of energy required to melt one kilogram of the material, and the Duration Index [measured in Joules/(cubic centimeter*degrees Centigrade)], which provides a basis of comparison of how long a PCM will remain at a constant temperature during its phase change.

Common PCMs include paraffins (alkanes), salt hydrates, eutectic compounds, fatty acids and esters (including animal fats) and others. Individual PCMs will suggest themselves over others depending on the user's specific requirements. Some transition sharply (at a given temperature), whereas others (especially with impurities) do so over a several degree temperature range with reduced heat capacity. Others lose the capacity to transition sharply after a certain number of uses (eutectics often degrade after a few thousand cycles, rendering them of little use in many applications). Some PCMs are highly flammable; some are not. In general, however, PCMs can be useful as thermal energy storage media provided their other chemical properties are consistent with a given application.

The disclosed system is an articulated joint treatment constant or phased temperature microclimate control system that can be fine-tuned at the time of manufacture to specific treatment/recovery applications, ambient temperature ranges and therapeutic regimens, and is capable of meeting either specific microclimate temperatures and/or cooling/heating periods. It does this while concurrently providing an adjustable fit and pressure to the joint, imparting protective force impact protection to the wearer, and while enabling either superior range-of-motion or fixed-position therapeutic treatment.

### BRIEF SUMMARY OF THE INVENTION

The present invention comprises a system and/or method that have one or more of the following features and/or steps, which alone or in any combination may comprise patentable subject matter.

According to one embodiment of the present invention, an articulated joint treatment system which creates a localized impact absorbing microclimate managed environment comprising one or more conforming wraps, these wraps being held in alignment or registration to each other via tangs, registration marks or other visible features used in combination with ties, straps or more solid mechanical connection; each wrap having one or a plurality of cells of one or more sizes and shapes wherein each of the one or plurality of cells is comprised of a momentarily deformable high modulus material containing cooling or heating thermal material, wherein the materials may be different from cell to cell.

In another embodiment of the present invention, the shape of the one or plurality of thermal material containment cells may be adjusted to be relatively flat on one face (which is worn against the treated area) with the opposing face being allowed to bulge outwards to contain a high volume of the cooling or warming material, in this manner creating a comfortable surface against the wearer's body while still incorporating long thermal treatment periods.

Another embodiment of the present invention may yet further comprise one or more sections which are capable of applying pressure to select portions of the wearer's body, said pressure being independently adjustable by section (chosen, for instance, by the cognizant healthcare professional or athletic trainer) and sustainable for extended periods of time.

In yet another embodiment of the present invention, said pressure-applying capacity may be configured as either a permanent feature of the system or one that is temporarily installed.

Yet still another embodiment of the present invention may further comprise an articulated joint treatment system wherein mechanical limiting structures, such as rigid or semi-rigid features are used in conjunction with holding pockets slits or other attachment structures known within the art to fix or immobilize the joint at a chosen angle.

A preferred embodiment will incorporate a constant or phased constant temperature microclimate control system to create a stable thermal environment, such as can be achieved by use of an appropriate PCM, which can be fine-tuned at time of manufacture.

### BRIEF DESCRIPTION OF THE DRAWINGS

FIGS. 1-4 depict multiple embodiments and configurations of treatment systems of the present invention.

FIG. 5 depicts one embodiment of tendon treatment within the scope of the present invention.

FIG. 6 depicts one embodiment of a hexagonal cell configuration of the present invention, wherein one face of the cells—which is worn against the patient—is adjusted to be relatively flat, while the opposing face is allowed to bulge outwards to contain a high volume of thermal treatment material.

FIG. 7 depicts one embodiment of joint immobilization within the scope of the present invention.

FIGS. 8-9 depict an embodiment and configuration having an integrated capacity to apply pressure that is within the scope of the present invention.

FIG. 10 depicts one embodiment and configuration within the scope of the present invention of a feature that can be installed into the articulated joint treatment system to apply pressure to select areas of the wearer.

### DETAILED DESCRIPTION OF THE INVENTION

Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

The invention is a ‘thermal management system’ comprised of one or several wraps, each manufactured to specific predetermined sizes/shapes for given articulated joint therapy applications. For any given case a wrap may be endothermic or exothermic (relative to nominal body temperature) with precise temperature(s) chosen at the time of manufacture to optimize therapeutic outcome. While this ‘thermal management system’ may be comprised of one or more pieces, one preferred embodiment uses from one to six pieces to treat an articulated joint—each installed to provide specific thermal management characteristics to and around the joint. This preferred embodiment includes one or multiple features on each piece/wrap to align or register the pieces to each other, so that when donned, the pieces maintain the best aspect/fit as determined by either the wearer or therapeutic provider. These alignment/registration features form an ‘active hinge’ between the pieces (holding wrap-to-wrap alignment as set when the system is first donned, ensuring that thermal treatment is optimally applied) enabling joint flexure, and may be manifest as a manufactured feature of one or more of the wrap pieces (whether the feature attaches directly or is held in place using various attachments) and/or may optionally consist of carefully designed ‘straps’ to interconnect the wrap pieces in a controlled manner. Such straps may be comprised of any suitable material/dimension that affords proper strength, stiffness, physical robustness, stretchiness (or lack thereof) and capability of being attached to the wrap(s).

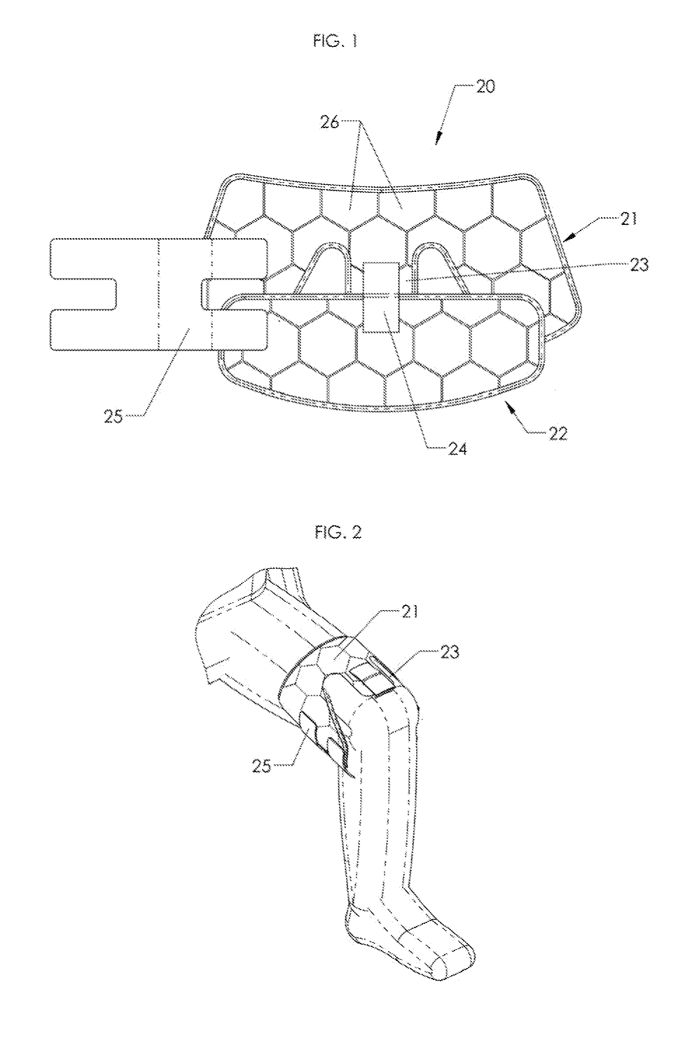

Referring to the drawings now in detail, reference is first made to FIGS. 1-4, wherein an articulated joint treatment system for knees is shown, generally designated by the numeral 20.

One embodiment of the “active knee” system 20 may comprise an upper wrap 21 and a lower wrap 22 that are registered via tang 23 which forms an “active hinge”, which is itself connected to lower wrap 22 via attachment strap 24. Note the upper wrap 21 has two wings pointing downward, which, when wrapped on the knee, direct thermal therapy to tears of the menisci and/or ligament damage, giving the wearer significant pain relief.

Each wrap is itself comprised of multiple layers, which may include: flexible, resilient, impact-resistant layers of tri-polymer plastic, TPU, or other urethane or polymer films, or other such materials exhibiting superior physical ‘memory’, strength and workability, formed into either one or a plurality of impact resistant cells/pockets of one or many shapes and thicknesses, themselves designed and specifically placed to balance the characteristics of physical flexibility and fluidity of movement, comfort, volumetric capacity, force impact protection performance, anti-microbial performance, durability and cleanability; cooling or warming material; and (optionally) other layers such as Veltex® display loop or other hook and loop attaching system and/or materials, enabling the use of various anchor attachment straps or other mechanical mounting and holding provisions.

Straps 24 and 25 (or like equivalent structures) are suitable for use in conjunction with attachment materials (such as the open loop configuration already described), such straps being comprised of stretchable and/or non-stretchable materials in sizes/shapes to enable proper fit and tension adjustment and to best enable comfort for the wearer.

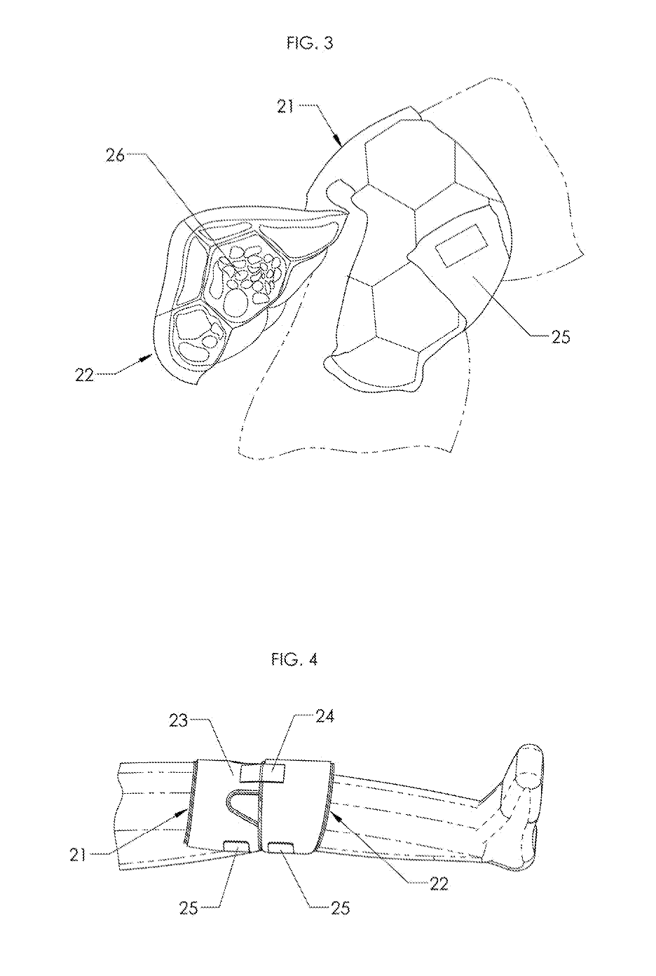

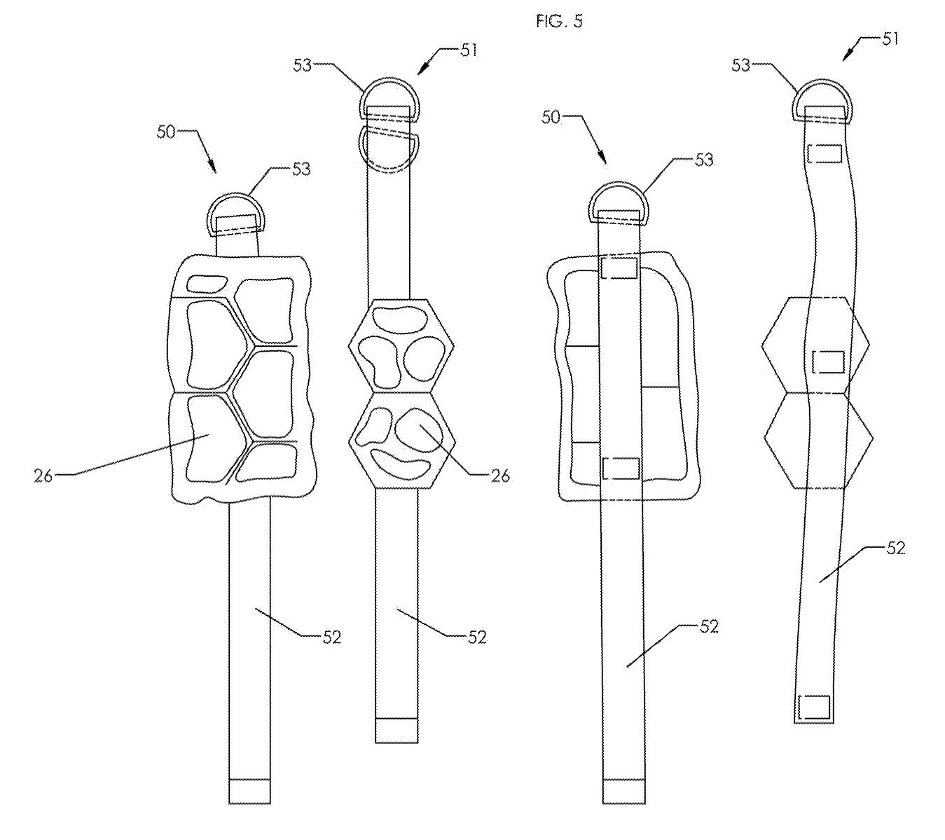

FIG. 3 depicts lower wrap 22 being “turned out” to show the inside surface, which (in this embodiment) is formed into hygienic thermal containment cells 26. FIG. 4 shows the active knee system 20 installed on a knee.

Reference is made to FIG. 5, which depicts additional wrap embodiments of the present invention that can be used in conjunction with articulated joint treatment systems 20 of the present invention. Such embodiments, as depicted at 50 and 51, are typically placed to treat specific muscles or tendons that may not be fully covered by a given joint wrap treatment system. Embodiments 50 and 51 are comprised of multiple material layers and include thermal material containment cells as described above herein, but also include mounting straps 52 terminated in tie down (or “D”) rings 53 which allow the straps 52 to be used on limbs of different sizes. Straps 52 are bonded to containment cells 26 via thermal sealing, RF sealing or other means to create a high-strength, low profile connection points that will not irritate the wearer's skin. Critical placement of these wraps (relative to other wraps pieces of an articulated joint treatment system) is accomplished by using a short (generally inflexible) strap such as that depicted as attachment strap 24 above to specifically register embodiments 50 or 51 or the like relative to other wraps.

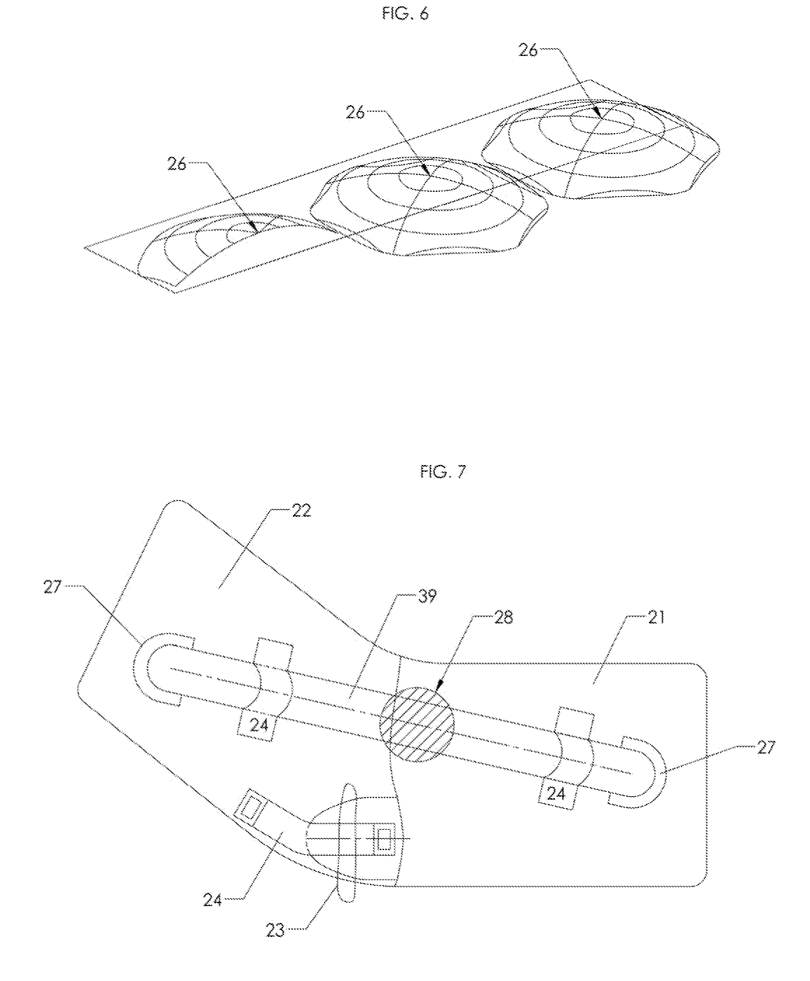

FIG. 6 depicts a cutaway view of contiguous thermal containment cells 26, herein shaped as hexagons of equal size. By controlling the bonding tension between the conjoined upper layer of display loop (or other material) and the layers of high modulus plastic, it is possible to force the containment cell face opposite the display loop into a relatively flat aspect—which is placed against the wearer's skin—while the layer conjoined with display loop bulges outwards to contain relatively high volumes of thermal PCM material. This feature is critical to enhancing wearer comfort, as containment cells are normally fully symmetric and can, when tightly filled, create uncomfortable “bulges” against the wearer when the PCM is charged. Relatively greater cell containment capacity translates to greater thermal treatment durations. Surrounding individual cells with thin peripheral transition spaces enables wrap flexibility so the wrap can conform to the wearer's limb/joint and will not hinder joint flexure.

Reference is made to FIG. 7, wherein another embodiment of the invention adds one or more mechanical limiting structures such as rigid features to the articulated joint treatment system 20 (in this case, to either side of the knee) to temporarily hold the articulated joint in a specific aspect/position. In this example, rigid feature 39 is inserted into and held in alignment by one or more retaining structures such as pockets 27 which may be a part of upper wrap 21 and lower wrap 22. A series of such pockets 27 (or slits) may be built into wraps 21 and 22 to facilitate specific rigid feature 39 positioning, and/or rigid feature 39 may include an adjustable center pivot 28 that may be set by the treatment professional. Additional straps, such as attachment strap 24, can be used to further stabilize rigid feature 39. One or more retaining structures such as pockets 27 are preferably stitched to their respective wrap, but the scope of the present invention includes one or more retaining structures including but not limited to stitching, hook and loop fasteners, rivets, slits, and the like.

One material particularly well suited as the rigid feature 39 of this application is polyethylene terephthalate (also known commercially as PET, PETE or PET-P), a thermoplastic polymer that can be formed into nearly any shape. Another well-suited material is Delrin®, a DuPont thermoplastic polymer. In a preferred embodiment, such a material is formed into a rod/shape whose dimensions make it possible to firmly ‘capture’ or hold this feature by two of the permanent pocket 27, slits, or other end attachment means. In yet another preferred embodiment, Velcro® or another display hook and loop compatible material is permanently affixed to the rod/shape (which may be held between or in the pockets 27), so when it touches the one or more articulated joint treatment wraps 21 and 22 (e.g. between the pockets) it provides further attachment strength, and providing a firm fixed position. By optionally using rods/shapes of different length in conjunction with different fixation elements on the wraps (including the use of threading on the rod and adjustment via secondary structures which are held in place on the treatment structure) the immobilized position/angle of the joint can be modified.

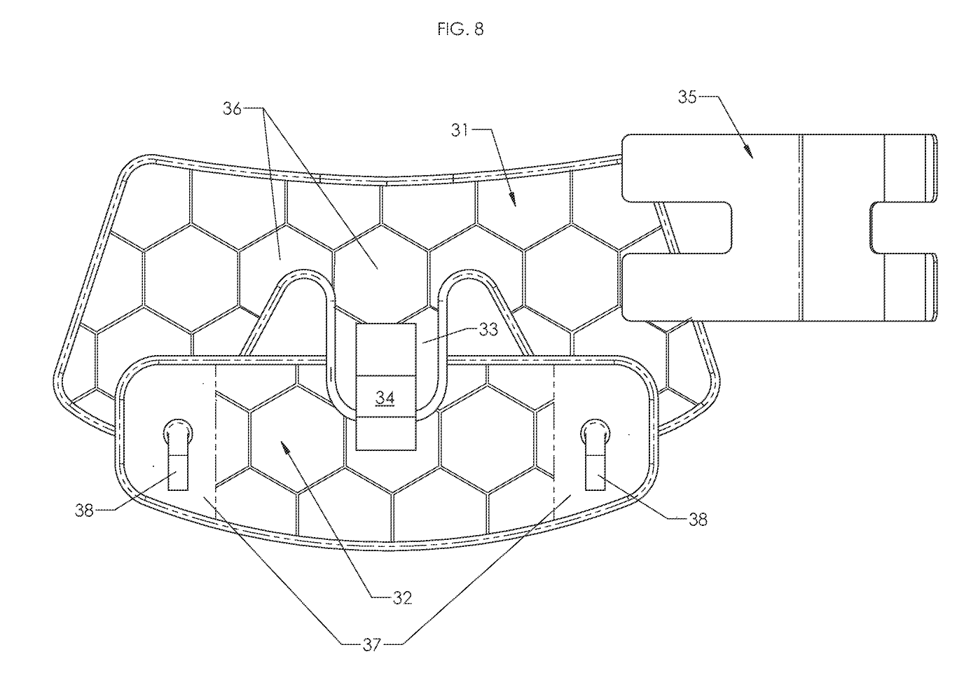

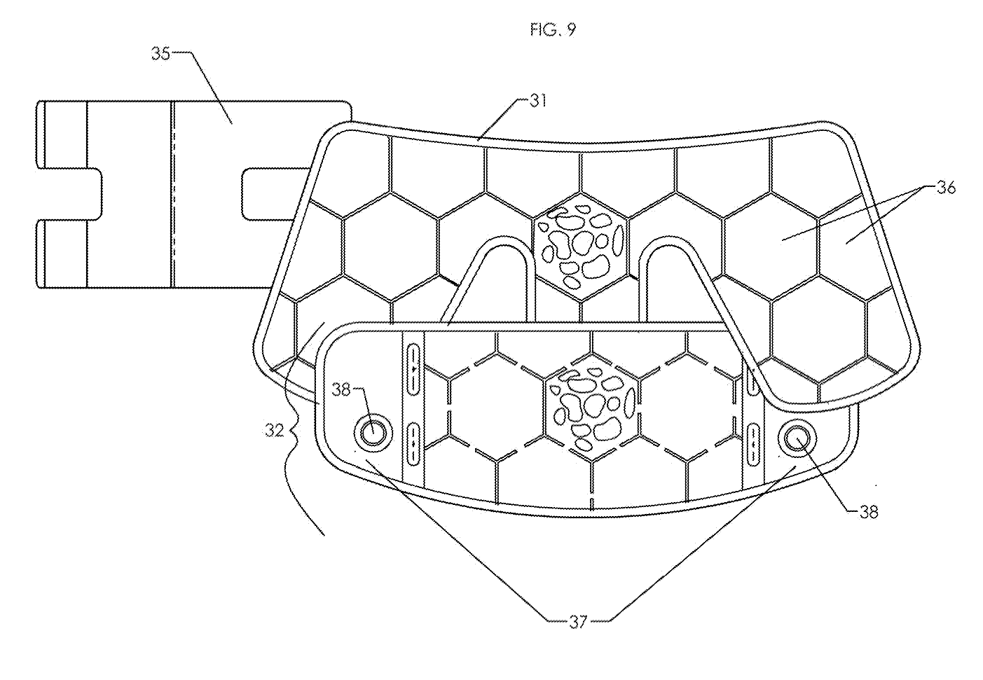

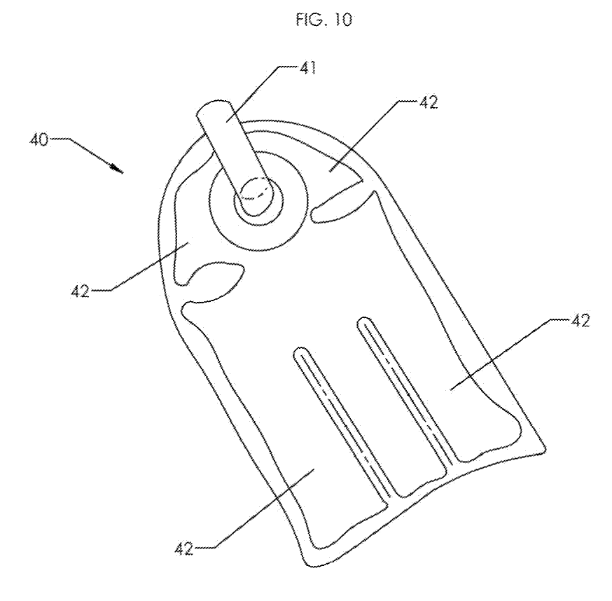

Reference is made to FIGS. 8-9, wherein yet another embodiment of a system 30 of the invention incorporates the ability to apply pressure to select portions of the wearer's limb. Lower wrap 32 is similar to lower wrap 22 except it incorporates sealed pressure bladders/cells 37 at either end of the wrap (positioning is representative and not restrictive). In FIG. 8 a complete pressure bladder 37 is shown in full aspect, consisting of a sealed cell with a valve 38. In this embodiment, when system 30 is donned, the wings of upper wrap 31 and the containment cells 36 are preferably placed directly against the body and lower wrap 32 is placed over these wings (being registered by the active hinge formed by tang 33 [not shown but same as tang 23 in FIG. 2] and attachment strap 34) and is held in place by strap 35 (such as with strap 25 shown in FIGS. 1 and 4). Valves 38 are used to inflate pressure cells 37 which develop pressure against the body. Individual pressure cells 37 can be pressurized with either liquid or gas and can be independently inflated (to different chosen pressures). The invention allows for incorporation of one or a plurality of pressure cells/bladders 37 that are used to apply pressure in a precise manner. Pressure cells 37 can be permanently integrated into a wrap's construction or may be temporarily accommodated by the articulated joint treatment system.

Reference is made to FIG. 10, depicting a single air pressure bladder 40, an embodiment of the present invention, with application for temporary inclusion in the articulated joint treatment system. Bladder 40 is comprised of the same high modulus materials used in constructing bladder(s) 37 and uses the same/equivalent valves 41 as those depicted in FIGS. 8-9 at valve 38. In a preferred embodiment, inclusion of sealed features 42 (flattened sections, which are exemplary but not restrictive of such shaping features) in the plastic layers provide specific shape to the bladder when pressurized. Such sealing features 42 are used both to reinforce bladder strength (note features around valve 41, for instance) and to precisely shape where/how pressure is applied to the wearer.

In one embodiment, provisions for temporarily including such a pressure bladder 40 into the articulated joint treatment system are made by inserting the bladder(s) 40 into bladder pocket(s) on the wrap's outer cloth or display loop layer (the bladder pocket is preferably stitched to this layer wrap, but the scope of the present invention includes all fixation means known within the art including but not limited to stitching, hook and loop fasteners, rivets, and the like) or by placing this bladder 40 between or under wrap surfaces (giving the treatment professional an opportunity to apply both pressure and thermal treatment in a precise way). Such a structure is capable of holding substantial—and sustained—pressure, with the pressure level and placement chosen by the treatment professional.

In a preferred embodiment, pressure valves 38 or 41 are incorporated into air pressure bladders 37 or 40 so they can be sealed to one of the high modulus layers comprising the wraps/bladders. A preferred embodiment uses a polyurethane “Schrader-like” or “Presta-like” valve featuring a flattened base held between layers of the high modulus urethane plastic. In this embodiment, a layer of TPU is permanently bonded to the valve base via RF welding, creating a reliable leak-free bond. In a preferred embodiment, the Schrader-like valve can be inflated or deflated via a handheld squeezable pressure bulb, facilitating easy adjustment of the pressure level.

In a preferred embodiment of the invention, the cooling or warming material is an alcohol based gel mixture directly confronting or encapsulating an electronic cooling or warming module, such as a miniature Peltier module, which holds the gel at a constant temperature. In another preferred embodiment, the cooling or warming material is a PCM incorporating a single phase transition point. In yet another embodiment, with appropriate design or formulation, the cooling or warming material is a PCM offering two to ten specifically chosen stable temperature transition points, and in yet another embodiment a single transition temperature which cannot be achieved by commonly available PCMs. In this way an articulated joint treatment system can create a specific microclimate phasing which, itself, depends on both ambient and wearer conditions. In still another preferred embodiment, the system's one or more wraps are comprised of a specifically chosen PCM or system of PCMs (with one or more stable temperature phase points) contained in a plurality of cells, said cells being confronted by a contained layer of gel which is worn directly against the body. In this way the gel can achieve the exact microclimate temperature(s) of the PCM(s), while providing a soft interfacing layer to the wearer. Preferably the cell-contained PCM is formulated or selected to yield phase transition points to within ±1° F. to ±10° F. of a targeted transition temperature. Other properties of specific PCMs that will enhance utility in an articulated joint treatment system featured in other embodiments include: (1) the capacity to undergo any number of thermal cycles without notable degradation, (2) the capacity to provide a relatively constant temperature microclimate for a long duration, (3) the chemical property of not being harmful or caustic to skin or organs and (4) third-party registration of such safety [such as an FDA 510(k)] and (5) dielectric properties that render the PCM non-conducting (enhancing user safety if the invention is used around electronics or electrical equipment). One such family of PCMs is HTFEXOTHERM® manufactured by HTFx Inc.

When using a somewhat or relatively viscous PCM such as HTFEXOTHERM®, whether in the somewhat hardened charged state or even in the fully discharged liquid state, the PCM material contributes directly to the wrap's ability to dissipate or dampen incident force impact energy, helping to protect the wearer from incident blunt force trauma. In preferred embodiments, these protective characteristics are dramatically amplified by containing the PCM in one or a plurality of cells that are built from physically malleable materials such as tri-polymer films or in a plurality of such cells designed to release or transfer PCM fluid from cell to cell when one or more cells experiences the high hydroscopic pressure caused by a force impact. This quality can be obtained by varying cell shape, size and placement, and by selecting one or more material sealing technologies—such as thermal impulse sealing and RF sealing—and by including one or a plurality of channels of one or many widths between cells (whether these channels are always open or some are forced open only by a high impact force) to adjust the strength and reactivity of the cell walls. This approach can be used quite effectively to create a system of small scale “baffles” from cell to cell, making it possible to disburse and dampen force impact shock waves across a plurality of cells and to create a system responsive to both high and low speed force impacts. Such baffling can be crafted to keep the articulated joint treatment system from applying greater than therapeutic levels of pressure to a wearer if the system is misused or improperly installed. These macroscopic qualities are maintained even when an incident blow has enough force to damage one or more cells. Absent a force impact, the PCM will remain relatively static in the plurality of cells. While such incident impact forces are unlikely in most articulated joint therapeutic settings, this design feature will protect the wearer who exercises using external equipment or suffers an unintended accident. Such baffling will also facilitate use of thermal management structures in association with joint immobilization or “air casts” by reducing the likelihood that a multi-cell structure will impose overly high, localized pressures.

Different embodiments will use different combinations of film material and number of layers, shape (including the use of one or many different wrap shapes simultaneously), size (including the use of one or many sizes simultaneously), placement of cells, size and shape of cells (including an intermixing of suitable and different cell sealing technologies to form effective systems of force dispersal, pressure applying, thermal treatment and dampening which can be incorporated into the articulating joint treatment system).

For instance, in a preferred embodiment, the plurality of impact resistant cells/pockets of one or many shapes and thicknesses are formed into hexagons, each hexagonal cell containing PCM material, such as containment cells 26 and 36 depicted in FIGS. 1-6 and FIGS. 8-9, respectively. In yet another preferred embodiment these hexagonal containment cells allow liquid or semi-liquid PCM to flow between cells when subjected to a given amount of pressure or flexing of the wrap. The hexagonal shape is particularly effective at balancing flexibility and fluidity of motion (necessary for comfort) with PCM capacity—enabling wraps that are capable of comfortably cooling/warming the affected joint for an extended duration.

Regarding the protective or microclimate utility or overall performance of the articulated joint treatment system, it is possible to either loosely or tightly couple each cooling or heating wrap around the wearer's articulated joint with specific fit/placement and tension adjusted for each wearer and to separately align multiple wraps with respect to each other in a preferred relation/fit using the one or many active hinge feature(s) formed by tang 23 and attachment strap 24 or tang 33 and attachment strap 34, respectively. This is particularly important when the wearer is participating in an ‘active’ exercise, range of motion or joint flexure regimen, to minimize the tendency of the one or more wraps to bunch up or move elsewhere on the wearer's limb (when referenced to the affected joint)—and to continue to deliver optimum thermal performance even during exercise.

In yet another variation of the immobilizing articulated joint treatment system, a device resembling any kind of air cast can be differently constructed to provide thermal treatment benefits by having one or more of its air bladders replaced by one or more thermal or thermal/pressure-applying wraps of suitable size and shape, the wraps being comprised of one or a plurality of cooling/heating cells containing suitable PCM material, or a combination of one or a plurality of thermal/pressure-applying cells such that the cast provides joint immobilization while the one or more wraps provides directed heating/cooling and/or pressure.

A number of embodiments of articulated joint treatment devices are detailed within this document, which also contains images of certain embodiments that have been reduced to practice. Other objects of the present invention will become apparent to those skilled in this art, including variations applicable to all other articulated body joints. As it will be realized, the invention is capable of other different embodiments and its several details are capable of modification in various, obvious aspects all without departing from the invention. The principles and concepts disclosed herein may easily and readily be applied to systems covering other joint and/or non-joint body surfaces and all such embodiment are within the scope of the present invention. Accordingly, all drawings, descriptions, embodiments, and specifically depicted examples will be regarded as only illustrative in nature and not as restrictive.

What is claimed is:

1. An articulated joint treatment system supportive of natural joint motion for maintaining at least one chosen constant temperature without connection to ancillary air or fluid systems or external power, comprising:
one or more containment cells having cooling or warming material therein;
one or more treatment wraps, wherein said one or more containment cells are disposed on said one or more treatment wraps; and
at least one strap for maintaining the position of said one or more treatment wraps.

2. The articulated joint treatment system of claim 1, wherein one or more treatment wraps comprise:
an upper wrap having at least one tang; and
a lower wrap having at least one attachment strap;
wherein said upper wrap and said lower wrap are connected via said at least one tang and said at least one attachment strap for forming a joint capable of articulation.

3. The articulated joint treatment system of claim 1, wherein one or more treatment wraps comprise:
an upper wrap having at least one attachment strap; and
a lower wrap having at least one tang;
wherein said upper wrap and said lower wrap are connected via said at least one attachment strap and said at least one tang for forming a joint capable of articulation.

4. The articulated joint treatment system of claim 1, wherein one or more treatment wraps comprise at least two layers of momentarily deformable, high modulus material, optionally sealed to one or more layers of cloth or display loop material, said at least two layers of momentarily deformable, high modulus material formed into said one or more containment cells containing said cooling or warming material therein.

5. The articulated joint treatment system of claim 1, wherein said cooling or warming material within said one or more containment cells is selected from the group consisting of alcohol based gel and a thermal energy module; Phase Change Material (PCM); PCM and thermal energy module; PCM and alcohol based gel; and PCM, thermal energy module, and alcohol based gel for effecting constant temperature cooling or warming, wherein said cooling or warming material is non-toxic and hypoallergenic.

6. The articulated joint treatment system of claim 1, wherein said one or more containment cells comprise a first face that is relatively flat for providing contact with a treatment area and a second face that is capable of bulging outwards to contain a high volume of said cooling or warming material.

7. The articulated joint treatment system of claim 1, wherein said system further comprises:
at least one pressure bladder in communication with said one or more treatment wraps, wherein at least one wrap cell is capable of applying select pressure to the wearer's body via temporary expansion and responsive tension of said at least one pressure bladder, said expansion being accomplished by adjusting pressure within said at least one pressure bladder via at least one valve, said pressure capable of being independently adjusted and sustained.

8. The articulated joint treatment system of claim 7, wherein said at least one pressure bladder further comprises one or more sealing features capable of controlling the inflated shape of said at least one pressure bladder to more precisely control the application of pressure.

9. The articulated joint treatment system of claim 7, wherein said at least one pressure bladder is integrally formed with said one or more treatment wraps.

10. The articulated joint treatment system of claim 7, wherein said at least one pressure bladder is in releasable communication with said one or more treatment wraps

11. The articulated joint treatment system of claim 2, further comprising:
one or more mechanical limits, wherein said one or more mechanical limits may comprise a rigid feature or a non-rigid feature; and
one or more retaining structures, said one or more retaining structures are selected from the group consisting of pockets, stitching, hook and loop fasteners, rivets, and slits;
wherein said one or more mechanical limits and said one or more retaining structures are used in combination to fix or immobilize said articulated joint treatment system at any chosen angle as said one or more mechanical limits are held in position by said one or more retaining structures.

12. The articulated joint treatment system of claim 3, further comprising:
one or more mechanical limits, wherein said one or more mechanical limits may comprise a rigid feature or a non-rigid feature; and
one or more retaining structures, said one or more retaining structures are selected from the group consisting of pockets, stitching, hook and loop fasteners, rivets, and slits;
wherein said one or more mechanical limits and said one or more retaining structures are used in combination to fix or immobilize said articulated joint treatment system at any chosen angle as said one or more mechanical limits are held in position by said one or more retaining structures.

13. The articulated joint treatment system of claim 5, wherein said PCM undergoes a phase change within the range of ±3° F. of its targeted phase temperature.

14. The articulated joint treatment system of claim 5, wherein said PCM undergoes a phase change within the range of ±1° F. of its targeted phase temperature.

15. The articulated joint treatment system of claim 1, wherein said one or more containment cells are contiguous.

16. The articulated joint treatment system of claim 1, wherein said one or more containment cells are non-contiguous.

17. An articulated joint treatment system supportive of natural joint motion for maintaining at least one chosen constant temperature without connection to ancillary air or fluid systems or external power, comprising:
an upper wrap;
a lower wrap in flexible communication with said upper wrap;
one or more containment cells having cooling or warming material therein disposed on each of said upper wrap and said lower wrap, wherein said one or more containment cells comprise a first face that is relatively flat for providing contact with a treatment area and a second face that is capable of bulging outwards to contain a high volume of said cooling or warming material, said cooling or warming material within said one or more containment cells is selected from the group consisting of alcohol based gel and a thermal energy module; Phase Change Material (PCM); PCM and thermal energy module; PCM and alcohol based gel; and PCM, thermal energy module, and alcohol based gel for effecting constant temperature cooling or warming, wherein said cooling or warming material is non-toxic and hypoallergenic; and
at least one strap for maintaining the position of said upper wrap and said lower wrap;
wherein said upper wrap and said lower wrap comprise at least two layers of momentarily deformable, high modulus material, optionally sealed to one or more layers of cloth or display loop material, said at least two layers of momentarily deformable, high modulus material formed into said one or more containment cells containing said cooling or warming material therein.

18. The articulated joint treatment system of claim 17, wherein said flexible communication between said upper wrap and said lower wrap comprises the meeting of at least one tang and at least one attachment strap for forming a joint capable of articulation between said upper wrap and said lower wrap.

19. The articulated joint treatment system of claim 17, further comprising:
one or more mechanical limits, wherein said one or more mechanical limits may comprise a rigid feature or a non-rigid feature; and
one or more retaining structures, said one or more retaining structures are selected from the group consisting of pockets, stitching, hook and loop fasteners, rivets, and slits;
wherein said one or more mechanical limits and said one or more retaining structures are used in combination to fix or immobilize said articulated joint treatment system at any chosen angle as said one or more mechanical limits are held in position by said one or more retaining structures.

20. The articulated joint treatment system of claim 17, wherein at least one pressure bladder in communication with said one or more treatment wraps, wherein at least one wrap cell is capable of applying select pressure to the wearer's body via temporary expansion and responsive tension of said at least one pressure bladder, said expansion being accomplished by adjusting pressure within said at least one pressure bladder via at least one valve, said pressure capable of being independently adjusted and sustained.
